(19) United States
(12) Patent Application Publication
(10) Pub. No.: US 2026/0183109 A1
(43) Pub. Date: Jul. 2, 2026

(54) DELIVERY DEVICE HAVING DYNAMIC FLEXIBLE SPINDLE

(71) Applicant: Medtronic Vascular, Inc., Santa Rosa, CA (US)

(72) Inventor: Christopher J. McCabe, Galway (IE)

(21) Appl. No.: 19/547,343

(22) Filed: Feb. 23, 2026

Related U.S. Application Data
(62) Division of application No. 17/910,658, filed on Sep. 9, 2022, now abandoned.
(60) Provisional application No. 62/988,636, filed on Mar. 12, 2020.

(51) Int. Cl. A61F 2/24; A61F 2/95; A61F 2/966

(52) U.S. Cl. CPC A61F 2/2439; A61F 2/2436; A61F 2002/9511; A61F 2002/9665

(57) ABSTRACT

The present disclosure provides a delivery device including an inner shaft assembly including an inner shaft having a proximal end and a distal end and a lumen. The inner shaft further includes a spindle connected to the distal end of the inner shaft and the spindle includes a body and a side lumen offset with respect to a central axis of the spindle. The delivery device further includes a spine wire that can slide within both the lumen of the inner shaft and the side lumen of the spindle. Additional lumens and spine wires can be provided. The disclosure further includes methods of using the delivery devices of the disclosure for delivery a stented prosthesis, for example.

### CROSS-REFERENCE TO RELATED APPLICATIONS

This application is a divisional of U.S. Application No. 17/910,658, filed September 9, 2022, which is the National Stage under 35 U.S.C. § 371 of International Application No. PCT/US2021/022082, filed March 12, 2021, which claims the benefit of U.S. Provisional Application No. 62/988,636, filed March 12, 2020, the entire contents of each application which are incorporated herein by reference.

### FIELD

The present technology is generally related to delivery devices for transcatheter delivery of a stented prosthesis.

### BACKGROUND

Diseased or otherwise deficient heart valves can be repaired or replaced with an implanted prosthetic heart valve. Conventionally, heart valve replacement surgery is an open-heart procedure conducted under general anesthesia, during which the heart is stopped and blood flow is controlled by a heart-lung bypass machine. Traditional open surgery inflicts significant patient trauma and discomfort, and exposes the patient to a number of potential risks, such as infection, stroke, renal failure, and adverse effects associated with the use of the heart-lung bypass machine, for example.

Due to the drawbacks of open-heart surgical procedures, there has been an increased interest in minimally invasive and percutaneous replacement of cardiac valves. With percutaneous transcatheter (or transluminal) techniques, a valve prosthesis is compacted for delivery in a catheter and then advanced, for example, through an opening in the femoral artery and through the descending aorta to the heart, where the prosthesis is then deployed in the annulus of the valve to be restored (e.g., the aortic valve annulus).

A delivery device must often navigate through tortuous anatomy as it is tracked through the vasculature to the treatment site within the heart. The catheter may be navigated through various anatomical turns as it travels within the vasculature, including the sharp bend of the aortic arch.

The present disclosure addresses problems and limitations associated with the related art.

### SUMMARY

The techniques of this disclosure generally relate to transcatheter delivery devices and elements thereof. Embodiments of the disclosure include a dynamically flexible spindle that can be selectively stiffened or, alternatively, be made more flexible. In this way, the spindle can be very flexible while navigating an aortic arch and can be made more rigid during deployment of the prosthesis, which can improve prosthesis deployment accuracy.

In one aspect, the present disclosure provides a delivery device including an inner shaft assembly including an inner shaft having a proximal end and a distal end and one or more lumens. The inner shaft further includes a spindle connected to the distal end of the inner shaft and the spindle includes a body having one or more lumens, wherein the one or more lumens may be one or more side lumens offset with respect to a central axis of the spindle. The delivery device further includes one or more spine wires that can slide within both a lumen of the inner shaft and a lumen of the spindle.

In another aspect, the disclosure provides a method including providing a delivery device having an inner shaft assembly having a spindle supporting a stented prosthesis. The inner shaft assembly supports a first spine wire that can slide within a first lumen of the inner shaft assembly and the spindle includes a first lumen that can receive the first spine wire. The method includes transitioning the first spine wire from a retracted position in which a distal tip of the first spine wire is proximal with respect to the spindle to an advanced position in which the first spine wire is inserted within the first lumen of the spindle. The method may further include transitioning one or more additional spine wires from a retracted position in which a distal tip of the additional spine wires are proximal with respect to the spindle to an advanced position in which the additional spine wires are inserted within a lumen of the spindle.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of a delivery device for delivering a stented prosthesis.

FIG. 2A is a partial, schematic illustration of the delivery device of FIG. 1 having a prosthesis positioned over an inner shaft assembly; the stented prosthesis shown in an expanded state.

FIG. 2B is a schematic illustration of the delivery device of FIG. 2A having a stented prosthesis positioned over the inner shaft assembly; a plurality of elongated tension members compressing the stented prosthesis into a compressed state.

FIG. 3 is a front view of a stented prosthesis that can be used with the delivery devices disclosed herein.

FIG. 4 is a partial, schematic diagram of a delivery device having the stented prosthesis of FIG. 3 loaded thereon in which forces applied by elongated tension members to compress the stented prosthesis have deformed the flexible spindle.

FIG. 5A is a partial, schematic diagram of a delivery device having a spine wire proximally retracted from a spindle supporting the stented prosthesis of FIG. 3 so that the spindle is flexible.

FIG. 5B is a partial, schematic diagram of the delivery device of FIG. 5A in which the spine wire is distally advanced into the spindle to increase the rigidity of the spindle during compression of the stented prosthesis.

FIG. 6A is a partial, schematic diagram of a spindle of the disclosure.

FIG. 6B is a cross-sectional view of the spindle of FIG. 6A.

FIG. 7 is a cross-sectional view of an alternate spindle of the disclosure.

FIG. 8 is a cross-sectional view of yet another alternate spindle of the disclosure.

FIG. 9A is a cross-sectional view of an alternate spindle having two spine wires distally advanced within a body of the spindle.

FIG. 9B is a cross-sectional view of the spindle of FIG. 9A having the spine wires proximally retracted from the body.

FIG. 10A is a schematic illustration of an alternate spindle of the disclosure.

FIG. 10B is a cross-sectional view of the spindle of FIG. 10A.

FIG. 11A is a perspective view of an alternate spindle of the disclosure.

FIG. 11B is a cross-sectional view of the spindle of FIG. 11A.

FIG. 11C is a cross-sectional view of an alternate spindle, similar to that of FIG. 11A.

FIG. 11D is a cross-sectional view of an alternate spindle, similar to that of FIG. 11A.

FIG. 12 is a partial, schematic illustration of an alternate spindle having a body that is shown as transparent for ease of illustration.

FIG. 13 is a perspective view of a spindle secured to a key plate.

FIG. 14A is a cross-sectional view of the spindle of FIG. 13 interconnected to an inner shaft with a hub.

FIG. 14B is a partial, perspective view of the inner shaft of FIG. 14A in which a distal end of the inner shaft is omitted for ease of illustration.

FIGS. 15-16 are a perspective views illustrating an alternate hub that can be used to interconnect a spindle to an inner shaft.

FIGS. 17A is a front, distal-side view of a receiving surface of a hub.

FIG. 17B illustrates an opposing side of the receiving surface of FIG. 17A.

FIG. 18 is a flow chart illustrating example methods of the disclosure.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. As used herein with reference to a stented prosthetic heart valve, the terms "distal" and "outflow" are understood to mean downstream to the direction of blood flow, and the terms "proximal" or "inflow" are understood to mean upstream to the direction of blood flow. Although the present disclosure has been described with reference to various embodiments, workers skilled in the art will recognize that changes can be made in form and detail without departing from the spirit and scope of the present disclosure.

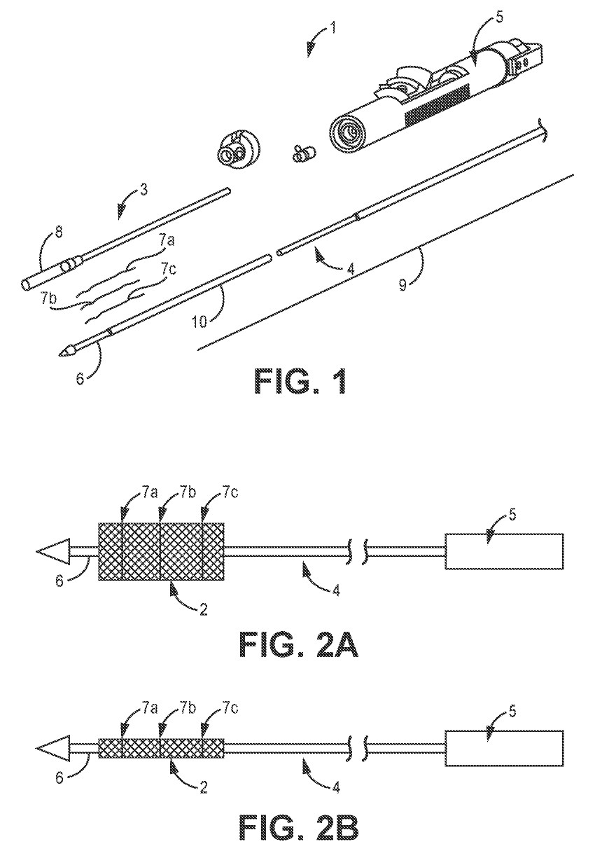

As described below, aspects of the present disclosure relate to delivery devices to deliver the stented prosthesis in a compressed state to a target site. By way of background, general components of one non-limiting example of a delivery device 1 with which the present disclosures are useful are illustrated in FIGS. 1-2B. The delivery device 1 is arranged and configured for percutaneously delivering a stented prosthesis 2, such as a prosthetic heart valve, to a patient’s defective heart valve. The delivery device 1 includes an optional outer delivery sheath assembly 3 including a capsule 8, an inner shaft assembly 4, and a handle assembly 5. The inner shaft assembly 4 can include an inner shaft 10 connected to a spindle 6. One or more elongate tension members 7a, 7b, 7c (schematically depicted) can optionally be provided, and can be considered part of the delivery device 1 in some embodiments or as part of the stented prosthesis 2 in other embodiments. The delivery device 1 provides a loaded delivery state in which the stented prosthesis 2 is loaded over the inner shaft assembly 4 and is compressively retained on the spindle 6 by the capsule 8 and/or the elongate tension members 7a-7c. In one example, the spindle is made of stainless steel or polyamide, for example. In one embodiment as is schematically illustrated in FIGS. 2A-2B, the compression on the stented prosthesis 2 is adjustable with one or more elongated tension members (e.g., sutures, cords, wires or the like) 7a-7c. Once the loaded and compressed stented prosthesis 2 is located at a target site, tension in the elongated tension members 7a-7c is lessened or released to permit the stented prosthesis 2 to self-expand, partially releasing and ultimately fully deploying the stented prosthesis 2 from the inner shaft assembly 4. In the illustrated embodiment, the optional delivery sheath assembly 3, where provided, can include the capsule 8 selectively disposed over the stented prosthesis 2 to assist in constraining the stented prosthesis 2 in the loaded or compressed state. The optional delivery sheath assembly 3 can be retracted by the handle assembly 5 to expose the stented prosthesis 2. In an alternative embodiment, the capsule 8 of the delivery sheath assembly 3 is disposed over the stented prosthesis 2 to fully constrain the stented prosthesis 2 in the loaded or compressed state. The optional delivery sheath assembly 3 is then retracted by the handle assembly 5 to release the stented prosthesis 2. The delivery device 1 can optionally be tracked over a guide wire 9 inserted through the handle assembly 5, inner shaft assembly 4 and spindle 6. Like reference numerals for components shown in FIGS. 1-2B will be used herein to identify identical components/structures.

As referred to herein, a stented prosthesis useful with the various devices and methods of the present disclosure may assume a wide variety of configurations, such as a bioprosthetic heart valve having tissue leaflets or a synthetic heart valve having polymeric, metallic or tissue-engineered leaflets, and can be specifically configured for replacing valves of the human heart. Although the stented prosthesis of the present disclosure is described mainly as being self-expandable, the stented prosthesis can also be balloon expandable and/or mechanically expandable or combinations thereof. In general terms, the stented prosthesis of the present disclosure includes a stent or stent frame having an internal lumen maintaining a valve structure (tissue or synthetic), with the stent frame having a normal, expanded condition or arrangement and is collapsible to a compressed condition or arrangement for loading within the delivery device. For example, the stents or stent frames are support structures that comprise a number of struts or wire segments arranged relative to each other to provide a desired compressibility and strength to the prosthetic valve. The struts or wire segments are arranged such that they are capable of self-transitioning from, or being forced from, a compressed or collapsed condition to a normal, radially expanded condition. The struts or wire segments can be formed from a shape memory material, such as a nickel titanium alloy (e.g., Nitinol). The stent frame can be laser-cut from a single piece of material, or can be assembled from a number of discrete components.

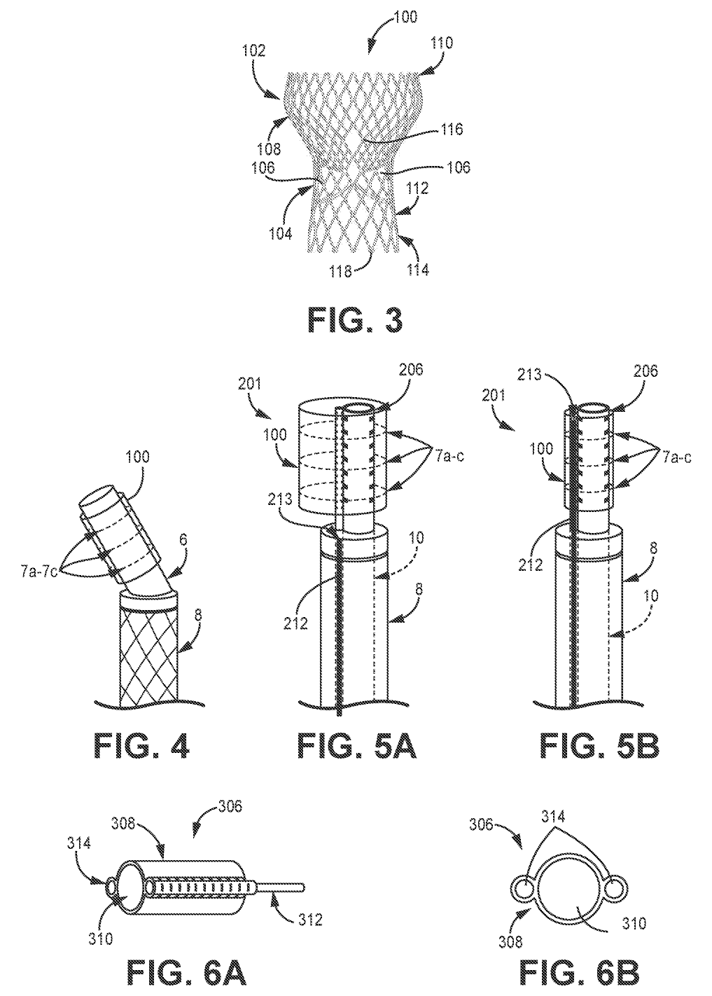

One simplified, non-limiting example of a stented prosthesis 100 is illustrated in detail in FIG. 3. It is to be understood that stented prosthesis 2 and stented prosthesis 100, referenced herein, are interchangeable. As a point of reference, the stented prosthesis 100 is shown in a normal or expanded state in the view of FIG. 3. The stented prosthesis 100 includes a stent or stent frame 102 and a valve structure 104. The stent frame 102 can assume any of the forms mentioned above. In some embodiments, the stent frame 102 is constructed to be self-expandable from the compressed state to the normal, expanded state. In some embodiments, the stent frame 102 is constructed to be balloon expandable from the compressed state to the normal, expanded state. In some embodiments, the stent frame 102 is constructed to be mechanically expandable from the compressed state to the normal, expanded state.

When present, the valve structure 104 of the stented prosthesis 100 can assume a variety of forms, and can be formed, for example, from one or more biocompatible synthetic materials, synthetic polymers, autograft tissue, homograft tissue, xenograft tissue, or one or more other suitable materials. In some embodiments, the valve structure 104 can be formed, for example, from bovine, porcine, equine, ovine and/or other suitable animal tissues. In some embodiments, the valve structure 104 can be formed, for example, from heart valve tissue, pericardium, and/or other suitable tissue. In some embodiments, the valve structure 104 can include or form one or more leaflets 106. For example, the valve structure 104 can be in the form of a tri-leaflet bovine pericardium valve, a bi-leaflet valve, or another suitable valve.

In some prosthetic valve constructions, such as that of FIG. 3, the valve structure 104 can comprise two or three leaflets that are fastened together at enlarged lateral end regions to form commissural joints, with the unattached edges forming coaptation edges of the valve structure 104. The leaflets 106 can be fastened to a skirt that in turn is attached to the stent frame 102. Alternatively, the leaflets 106 can be fastened directly to the stent frame 102. The stented prosthesis 100 includes an outflow portion 108 corresponding to a first or outflow end 110 (forcing out fluid) of the stented prosthesis 100. The opposite end of the stented prosthesis 100 can define an inflow portion 112 corresponding to a second or inflow end 114 (receiving fluid). As shown, the stent frame 102 can have a lattice or cell-like structure, and optionally forms or provides posts 116 corresponding with commissures of the valve structure 104 as well as eyelets 118 (or other shapes) at the outflow and inflow ends 110, 114. If provided, the posts 116 are spaced equally around frame 102 (only one post 116 is clearly visible in FIG. 3).

With many radial frame deployment delivery device designs, distal flexibility presents an issue in at least two ways. For one, when tracking the spindle around the aortic arch, the presence of the spindle in the delivery device limits the flexibility of the distal end of the delivery device. That said, having too flexible of a spindle impairs prosthesis deployment accuracy and the ability to effectively compress the stented prosthesis on the spindle with sutures or the like prior to delivery without deforming the spindle. As shown in FIG. 4, for example, if the flexible spindle 6 is not supported during loading of the stented prosthesis 100, the force exerted by sutures/elongated tension members 7a-7c used for compressively retaining the stented prosthesis 100 on the spindle 6 can cause deformation of the spindle 6. In other words, the spindle 6 may bend and/or be forced to an angle with respect to the inner shaft 10. Aspects of the disclosure include a spindle that can be utilized in a delivery device having a selectively variable stiffness. Delivery devices of the disclosure include one or more spine wires to provide selective rigidity to a spindle, such as spindle 6. As shown in FIGS. 5A-5B, embodiments of the disclosure provide for a delivery device 201 having selective rigidity in that one or more spine wires 212 can be proximally retracted from spindle 206 (so that a distal tip 213 is proximal with respect to the spindle 206) when flexibility of the spindle 206 is desired. The spine wire 212 can be distally advanced at least partially into the spindle 206 to provide greater rigidity in the spindle 6, when desired, such as during loading of the stented prosthesis 100. Therefore, each spine wire 212 is made of a material that is flexible but is more rigid than a material of the spindle 206. In some embodiments, one or more spine wires 212 are made of the same material and have the same flexibility or stiffness as the other spine wires 212. In some embodiments, one or more spine wires 212 are made of a different material and have a different flexibility or stiffness as the other spine wires 212. In some embodiments, one or more spine wires 212 have a different x-sectional shape and/or diameter as the other spine wires 212. For example, different x-sectional shapes may include round, oval, triangular, square, and/or hexagonal. The spindle 206 is provided in these illustrations as an example and it is to be understood that the other spindles disclosed herein will operate in a similar manner. It should also be understood that all spine wires disclosed herein are similarly configured and operate in a similar manner.

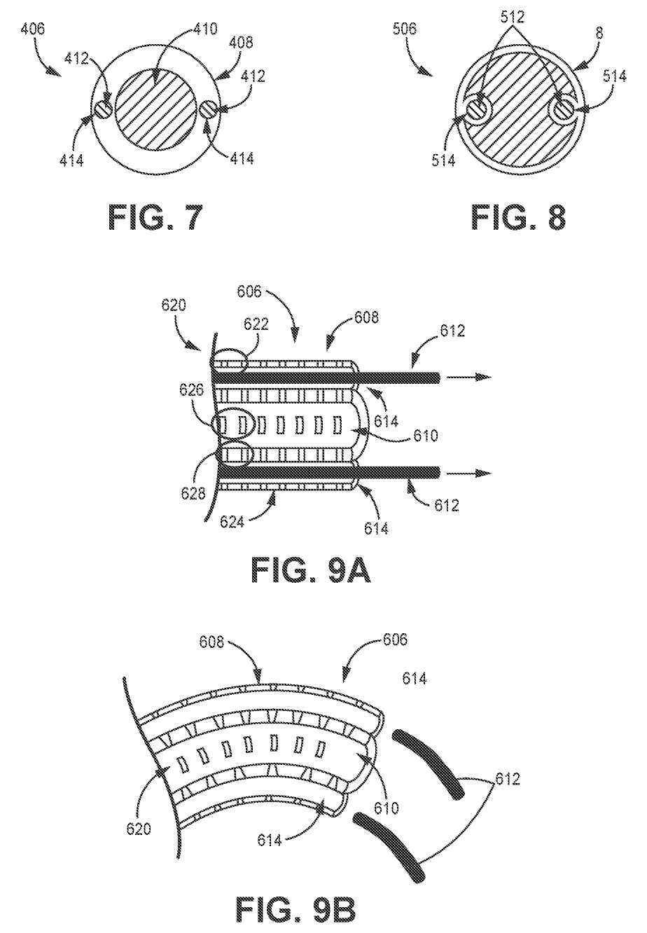

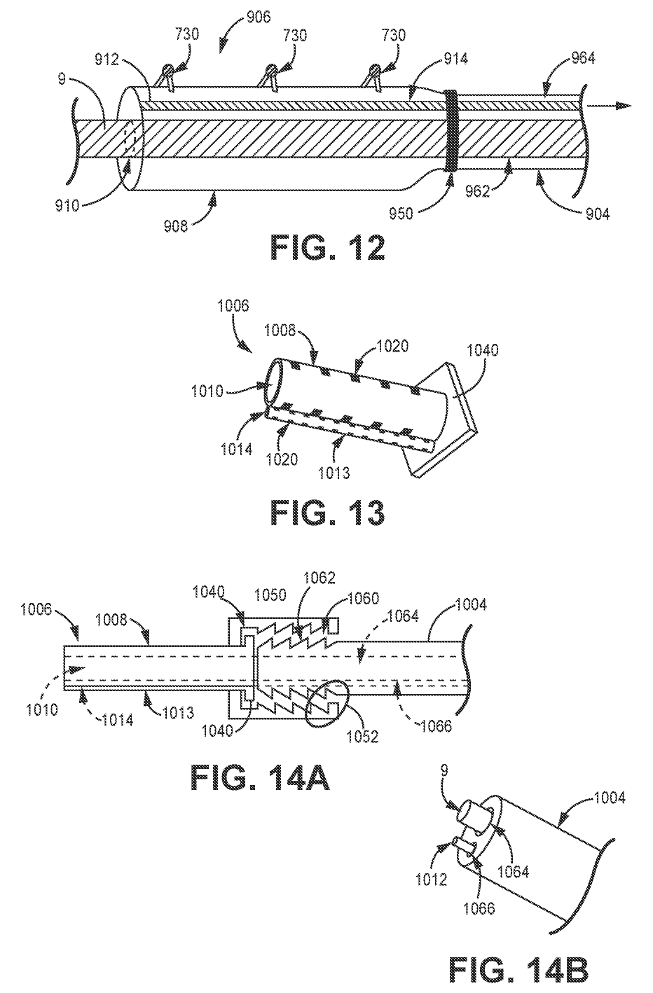

Referring now in addition to FIGS. 6A-6B, which illustrate part of a spindle 306 that can be used in a delivery device, such as a replacement for spindle 6 of FIGS. 1-2B, for example. In one example, the spindle 306 includes a body 308 optionally defining a central lumen 310 that can be used for tracing a guide wire 9 (see also, FIGS. 1 and 12). The body 308 can further define two or more additional side lumens 314 offset with respect to a central axis of the spindle 306. As shown, the side lumens 314 can be positioned outside of the body 308 and can be about 180 degrees (+/- 5 degrees) from each other with respect to a circumference of the body 308. One or more side lumens 314 can be configured to each receive one spine wire 312. Alternatively, as shown in FIG. 7, a spindle 406 can include a body 408 having a central lumen 410 and two side lumens 414 formed within the material of the body 408 and offset with respect to a central axis of the spindle 406, so that both the body 408 and the spindle 406 as a whole have a uniform outer diameter. Each side lumen 414 is configured to receive one spine wire 412. The spindle 406 of FIG. 7 can otherwise be identically configured and operate in an identical manner to spindles disclosed above. In yet another example shown in FIG. 8, a spindle 506 can include a body 508 that omits the central aperture and includes side lumens 514 at least partially formed within the body 508 and open to an outer covering,. Each side lumen can be offset with respect to a central axis of the spindle 506 and is configured to receive a spine wire 512. The spindle of FIG. 8 can otherwise be identically configured and operate in an identical manner to spindles disclosed above. It is to be understood that spindles 406 or 506 can be used as a replacement for spindle 6 in FIG. 1, for example.

The side lumens 314, 414, 514 can each receive a respective spine wire 312, 412, 512 inserted in a distal direction within one side lumen 314, 414, 514 from a proximal position with respect to the spindle 306, 406, 506 (i.e. such that a distal tip of the spine wire is proximal to the spindle). When inserted within the side lumen of the spindle (an “advanced position”), the spine wire provides greater stiffness to the spindle. When the spine wire is proximally retracted such that the distal tip of the spine wire is proximal to the spindle, the spindle has an increased flexibility. It is envisioned that any of the disclosed spindles can include additional side lumens and spine wires, as desired.

Referring in addition to FIGS. 9A-9B, in some embodiments, a body of any of the disclosed spindles can include a plurality of cuts (generally referenced) extending along a length of the body around its circumference configured to provide greater flexibly of the body in two planes. For example, a spindle 606 can have a body 608 including a plurality of cuts 620 (generally referenced). The plurality of cuts 620 can include a first set of cuts 622 (generally referenced) extending longitudinally in a row on one side of the body 608 and a second set of cuts 624 (generally referenced) extending longitudinally in a row on a second side of the body 608, 180 degrees from the first set of cuts 622. When spine wires 612 are inserted within their respective side lumens 614 (FIG. 9A), the plurality of cuts 320 restricts flexibility of the spindle 606 to one plane. As can be seen, the side lumens 614 are offset with respect to a central axis of the spindle 606. The plurality of cuts 620 can extend through the entire thickness of the body 608 or only part of the thickness of the body 608. The plurality of cuts 620 can be formed in many ways including laser cutting. In some embodiments, additional rows of cuts 626, 628 are provided. It will be understood that any of the spindle bodies disclosed herein can be configured to have a plurality of cuts 620 as disclosed with respect to FIGS. 9A-9B. To reduce the stiffness of the spindle 606, the spine wires 612 are proximally withdrawn from the side lumens 614 as shown in FIG. 9B.

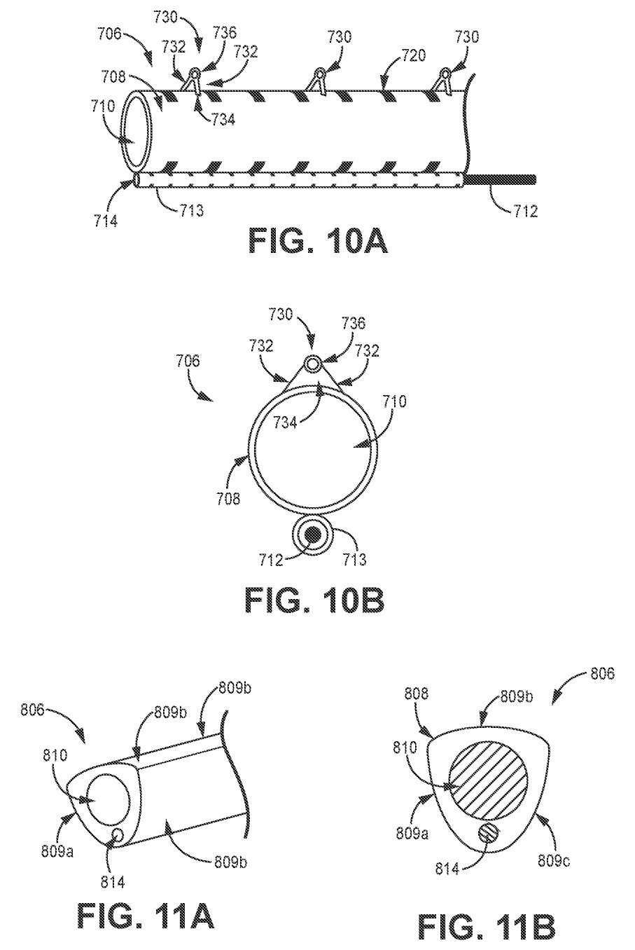

Referring in addition now to FIGS. 10A-10B, which illustrate yet another spindle 706 that can be incorporated into a delivery device, such as the delivery device of FIGS. 1-2B, as a replacement for spindle 6. The spindle 706 includes a body 708 having an optional central lumen 710 and one or more side lumens 714 offset with respect to a central axis of the spindle 706. In the embodiment of FIGS. 10A-10B, the side lumen 714 is formed outside of the body 708. For example, the side lumen 714 may be formed by a tube 713 bonded to the body 708. In some embodiments, the body 708 includes a plurality of cuts 720 (generally referenced) extending along a length of the body 708 around its circumference configured to provide greater flexibly of the body 708 in two planes as disclosed above. When a spine wire 712 is inserted within the side lumen 714, the plurality of cuts 720 restricts flexibility of the spindle 706 to one plane. The plurality of cuts 720 can extend through the entire thickness of the body 708 or only part of the thickness of the body. To reduce the stiffness of the spindle 706, the spine wire 712 is proximally withdrawn from the side lumen 714.

Any of the spindles of the present disclosure can optionally include one or more features 730 through which elongated tension members 7a-7c can be routed. In the example of FIGS. 10A-10B, a plurality of features 730 are secured on the body 708, opposite the tube 713. Each feature 730 can have two legs 732 defining an opening 734 through which one or more elongate tension members 7a-7c can be routed (see also FIGS. 1-2B). Opposite the body 708, the legs 732 are interconnected with an arcuate portion 736, such as a ball, for example. Generally, each feature 730 is configured to have rounded surfaces so that the elongated tension members are not abraded.

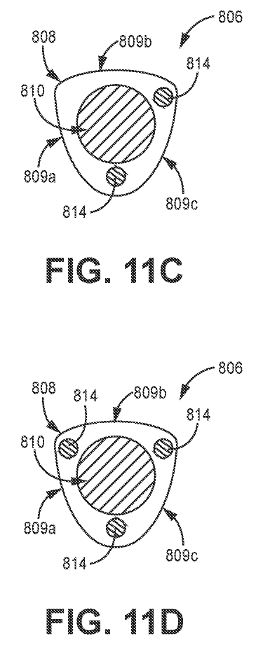

In other various embodiments, as shown in FIGS. 11A-11D, a spindle 806, such as any of those disclosed herein, can have a body 808 having a generally triangular cross-section (i.e., the body 808 is formed by three interconnected sides 809, 809b, 809c). The spindle 806 can have a central lumen 810 and one or more side lumens 814 formed within the body 808, the side lumens 814 offset with respect to a central axis of the spindle 806. As with prior disclosed embodiments, the side lumens 814 can receive a spine wire (similar to any disclosed herein) for selectively stiffening the spindle 806, as desired, in a manner disclosed herein.

Referring in addition to FIG. 12, which illustrates an alternate spindle 906 interconnected to an inner shaft 904 with a hub 950. It will be understood that the illustrated components can be used in a delivery device, such as that of FIGS. 1-2B as a general replacement for spindle 6 and inner shaft 10. In this embodiment, that spindle 906 includes a central aperture 910 through which a guide wire 9 can be inserted. The spindle 906 also includes a side lumen 914 through which a spine wire 912 can be optionally inserted to adjust rigidity of the spindle 906. In this example, the side lumen 914 is offset with respect to a central axis of the spindle 906. The inner shaft 904 includes a corresponding first aperture 962 aligned with and in communication with the central aperture 910 so that the guide wire 9 can be directed through the first aperture 962 to the central aperture 910. The inner shaft 904 also includes a second aperture 964 aligned with the side aperture 914 so that the spine wire 912 can be directed from the second aperture 964 to the side aperture 914. Both the spindle 906 and the inner shaft 904 can include additional apertures to accommodate additional spine wires or other components, as desired. Optionally, the body 908 can include one or more features 730, as discussed in more detail above. In one example, the spindle 906 and inner shaft 904 are interconnected with a hub 950. Additional disclosure relating to various hub configurations is provided with respect to FIGS. 14A-16.

Referring now in addition to FIGS. 13-14A, which illustrate an alternate spindle 1006 that can be used with the delivery device of FIGS. 1-2B as a replacement for spindle 6. The spindle 1006 includes a body 1008 defining a central lumen 1010. One or more side lumens 1014 are also defined and can be configured within the body 1008, within a separate tube 1013 bonded to the body 1008 or in any other way disclosed herein. In this example, the side lumens 1014 are offset with respect to a central axis of the spindle 1006. The spindle 1006 further includes a key plate 1040 connected to the body 1008. The body 1008 and/or tube 1013 can include a plurality of cuts 1020 (generally referenced) to impart flexibility in two or more planes as disclosed with respect to other embodiments. The key plate 1040 is configured to maintain alignment with an inner shaft 1004 (FIGS. 14A-14B, see also, FIG. 1 and related disclosure). In this way, the key plate 1040 has a square or other polygon cross-section as viewed perpendicular from a longitudinal axis of the spindle 1006. As shown in FIG. 14A, a hub 1050 is provided to interconnect the spindle 1006 to the inner shaft 1004. In one example, a distal end 1060 of the inner shaft 1004 includes a plurality of angled barbs 1062 that engage respective recesses 1052 in the hub 1050 via a push fit. In one example, the inner shaft 1004 may further be secured to the hub 1050 with adhesive applied to the barbs 1062. Other methods of fixedly securing the inner shaft 1004 to the hub 1050 are also envisioned. The hub 1050 further includes a recess 1054 configured to maintain the key plate 1040.

Referring now in addition to FIG. 14B, which illustrates the inner shaft 1004 of FIG. 14B in additional detail (the distal end 1060 of the inner shaft 1004 is omitted for ease of illustration). In this example, the inner shaft 1004 includes a first lumen 1064 and a second lumen 1066. Additional lumens can be provided, as desired to accommodate additional spine wires 1012 or other components. The first lumen 1064 can receive a guide wire 9 and is therefore aligned with and in communication with the central lumen 1010 of the spindle 1006. In the present example, the central lumen 1010 and the first lumen 1064 are interconnected via the hub 1050. The second lumen 1066 of the inner shaft 1004 can receive a spine wire 1012 and is in communication with the side lumen 1014 of the spindle 1006. In the present example, the second lumen 1066 and the side lumen 1014 are interconnected via the hub 1050. The inner shaft assembly 1004 and hub 1050 can be used with any of the spindles disclosed herein.

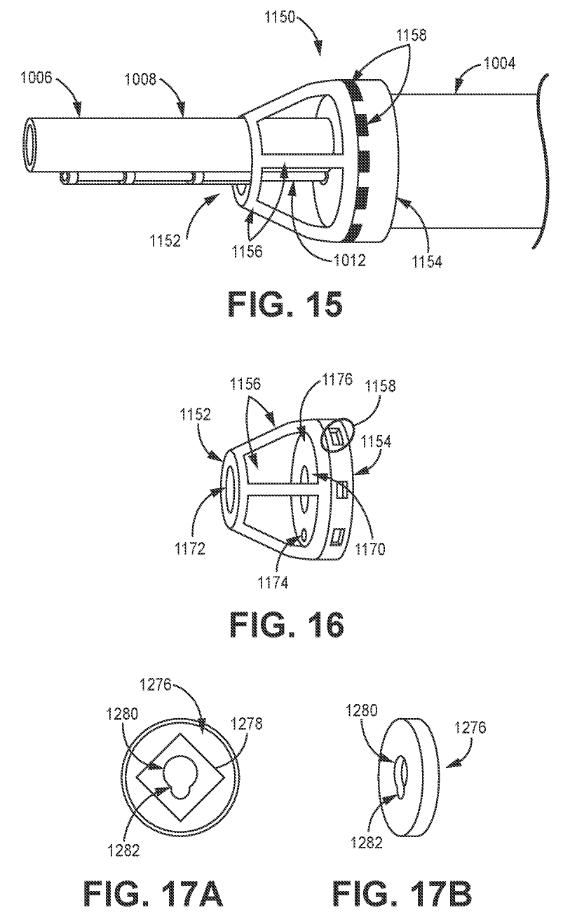

Referring now in addition to FIGS. 15-16, which illustrate an alternate hub 1150 that can be used to interconnect any spindle and inner shaft of the disclosure. In one example, the hub 1150 includes a distal collar 1152 interconnected to a proximal collar 1154 with a plurality of supports 1156 (only a few of which are referenced for ease of illustration). The distal collar 1152 can be spot welded or otherwise fixedly secured to the body 1008 of the spindle 1006. The proximal collar 1154 can be similarly spot welded or otherwise fixedly secured to the inner shaft 1004. The proximal collar 1154 can include one or more windows 1158 for reflow material. The proximal collar 1154 and the distal collar 1152 include respective apertures 1170, 1172 arranged and sized to receive the body 1008 of the spindle 1006. The proximal collar 1154 can include one or more additional apertures 1174 to receive addition components, such as the spine wire 1012 as is shown in FIG. 15. The spine wire 1012 can extend from the aperture 1174 through to the distal collar 1152 and out aperture 1172 or the distal collar 1152 can include a separate aperture for the spine wire 1012.

The hub 1150 of FIG. 16 can include a receiving surface 1176 configured to engage a key plate of the spindle 1006 (for example, see key plate 1040 and related disclosure) to maintain the orientation of the spindle 1006 with respect to the inner shaft 1004. In the example of FIGS. 17A, such a receiving surface 1276 includes an accepting slot 1278 corresponding in shape to a shape of the key plate of the spindle. In one example, the receiving surface 1276 can include one aperture 1280 interconnected to a second, smaller aperture 1282. The first and second apertures 1280, 1282 may be joined (FIGS. 17A) or they may have distinct and separate boundaries (as would coordinate with the hub of FIGS. 15-60). When the key plate (not shown) is inserted within the accepting slot 1278, the key plate cannot rotate with respect to the hub 1150 so that the alignment of the spindle with respect to the inner shaft is maintained. An opposing side, opposite the receiving surface 1276 is shown in FIG. 17B. It is to be understood that any of the hubs disclosed herein are suitable for use with any of the spindles, inner shafts and delivery devices disclosed herein.

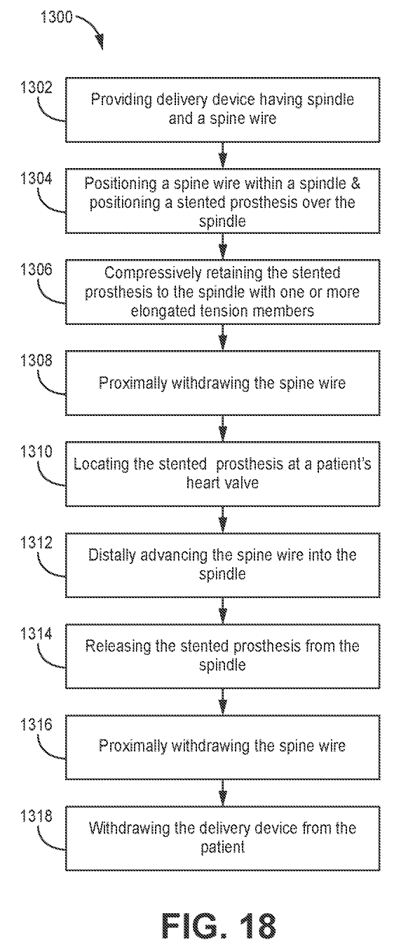

Methods of the disclosure are outlined in FIG. 18. One method 1300 of delivering a stented prosthesis includes providing a delivery device having a spindle and a spine wire 1302. The delivery device being of the type suitable for delivering a stented prosthesis to a target site via a transcatheter procedure. The stented prosthesis being any of the type disclosed herein. The method includes compressively retaining and securing the stented prosthesis to a spindle of the delivery device 1306. In one example, the stented prosthesis is compressively secured with one or more sutures (or alternate elongated tensioning members) while the spine wire is distally advanced within the spindle to support and provide rigidity to the spindle 1304/1306. Once the stented prosthesis is compressively retained on the spindle, the spine wire can be proximally withdrawn to a position proximal the spindle 1308. The tension in the elongate tension members can be loosened slightly, if desired. Then, the delivery sheath assembly can be positioned over the stented prosthesis. The delivery device is advanced using known techniques into a femoral artery of a patient and tracked around the patient’s aortic arch. The flexible capsule, cuts in the spindle, retracted spine wire and reduced tension in the elongated tension members provide flexibility at the stented prosthesis to provide easier navigation of the delivery device around the tortuous aortic arch. When the stented prosthesis is in position at a heart valve 1310, ready for deployment, the spine wire can be distally advanced into the spindle 1312, the elongated tension members can be further tensioned and the delivery catheter (which may or may not include the capsule) can be proximally retracted. Then, the tension in the elongated tension members can be released, which will allow the stented prosthesis to expand to its natural arrangement. When the stented prosthesis is in the desired position, the elongated tension members can be severed to deploy the stented prosthesis and release the stented prosthesis from the spindle 1314. The spine wire can be proximally withdrawn, proximal to the spindle so that the spindle is flexible again 1316. The elongated tension members are removed with the delivery device. Removal of the delivery device can include advancing the capsule to cover the spindle, retracting the spine wire and proximally withdrawing the delivery device along the path of delivery 1318.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method comprising:
providing a delivery device having:
an inner shaft assembly having a spindle supporting a stented prosthesis; wherein the inner shaft assembly supports a first spine wire that can slide within a first lumen of the inner shaft assembly; further wherein the spindle includes a first lumen that can receive the first spine wire; and
transitioning the first spine wire from a retracted position in which a distal tip of the first spine wire is proximal with respect to the spindle to an advanced position in which the first spine wire is inserted within the first lumen of the spindle.

2. The method of claim 1, further comprising proximally retracting the first spine wire proximally past the stented prosthesis and directing the stented prosthesis to a heart valve.

3. The method of claim 2, further comprising distally advancing the first spine wire into the first lumen of the spindle when the stented prosthesis is at the heart valve.

4. The method of claim 3, further comprising manipulating the delivery device to expand the stented prosthesis.

5. The method of claim 4, further comprising releasing the stented prosthesis from the delivery device and then proximally retracting the first spine wire from the spindle.

6. The method of claim 1, wherein the delivery device includes a second spine wire, the spindle includes a second lumen and the inner shaft assembly includes a second lumen, the second spine wire being positioned within the second lumen of the inner shaft assembly, the method further including transitioning the second spine wire from a retracted position in which a distal tip of the second spine wire is proximal with respect to the spindle to an advanced position in which the second spine wire is inserted within the second lumen of the spindle.

7. The method of claim 1, wherein the spindle includes a body having a plurality of cuts.

8. The method of claim 7, wherein the spindle includes a body supporting a feature extending from the body; wherein the stented prosthesis is compressively retained on the body with an elongate tension member that are routed through the feature.

9. The method of claim 8, wherein the feature includes two legs interconnected by an arcuate surface.
